(19) United States
(12) Patent Application Publication
(10) Pub. No.: US 2018/0147078 A1
(43) Pub. Date: May 31, 2018

(54) RUBBER CAP CATHETER FOR UNIFORM DEPLOYMENT OF SEGMENTED STENT

(71) Applicant: Cook Medical Technologies LLC, Bloomington, IN (US)

(72) Inventor: Woong Kim, West Lafayette, IN (US)

(21) Appl. No.: 15/725,533

(22) Filed: Oct. 5, 2017

Related U.S. Application Data
(60) Provisional application No. 62/427,876, filed on Nov. 30, 2016.

(51) Int. Cl. A61F 2/958; A61F 2/915

(52) U.S. Cl. CPC A61F 2/958; A61F 2/915; A61F 2002/828; A61F 2002/91583; A61F 2002/9583

(57) ABSTRACT

The present disclosure provides for an assembly and methods of use to deploy a segmented medical device to a body vessel. The assembly includes a multiportion medical device which has a plurality of detachable components, a balloon catheter, and a retention member at the distal end of the balloon of the balloon catheter so as to alter the inflation profile of said balloon, such that microsliding of the device over the exterior of the balloon is prevented or minimized.

### FIELD

The present disclosure relates generally to medical device assemblies. More specifically, the disclosure relates to an assembly and method(s) for delivering a multi-segment device, including but not limited to a stent, to a body vessel in order to treat a disease.

### BACKGROUND

Various implantable medical devices can be deployed within the lumen of a body vessel using minimally-invasive transcatheter techniques. Such devices can include an expandable frame configured for implantation in the lumen of a body vessel, such as an artery, a vein, or another body cavity. Minimally invasive techniques and instruments for placement of intraluminal medical devices have been developed to treat and repair undesirable conditions by implantation of the medical devices within body vessels. Intraluminal medical devices can be introduced to a point of treatment within a body vessel using a delivery catheter device passed through the blood vessels communicating between a remote introductory location and the implantation site, and released from the delivery catheter device at the point of treatment within the body vessel. Intraluminal medical devices can be deployed in a vessel at a point of treatment, the delivery device withdrawn from the vessel, and the medical device retained within the vessel to provide sustained improvement in vascular function or to increase vessel patency. Implantable medical devices can desirably comprise features that secure the implantable device within the body vessel upon deployment.

In certain circumstances, including in the treatment of disease conditions which present clinical situations in which a vessel of a patient needs to be artificially supported to maintain an open passageway through which fluids, such as blood, can flow, a certain region of a blood vessel may benefit from being kept open while still allowing flexibility during treatment. For example, blood flow through an artery can be impeded due to a build-up of cholesterol on the interior wall of the vessel. Also, vessel walls can be weakened be a variety of conditions, such as aneurysms.

Intraluminal support frames, sometimes referred to as stents, can provide an artificial mechanism to support a body vessel. Stents are typically tubular-shaped members that are placed in the lumen of the vessel and, once deployed, exert a radially-outward directed force onto the vessel wall to provide the desired support. Stents are typically positioned at the point of treatment or target site by navigation through the vessel, and possibly other connected vessels, until the point of treatment is reached. This navigation may require the stent to be able to move axially through the vessel(s) prior to deployment, while still maintaining the ability to exert an outward force on the interior wall once deployed. Accordingly, stents typically have radially unexpanded and expanded configurations. In the unexpanded configuration, the stent has a relatively small diameter that allows it to move axially through the vessel. In the expanded configuration, the stent has a relatively large diameter that allows it to exert an outward force on the interior wall of the lumen, thereby providing the desired support to the vessel.

In the case in which extra flexibility is desired, a segmented medical device such as a segmented stent may be employed. A segmented medical device may be delivered as a single interconnected unit, with its several separable members detaching from one another at the time of implantation in order to provide both the support of the conventional stent due to the radial stiffness of each segment and the increased flexibility that arises from separation of the portions.

Although the separable, segmented device may have advantages, the delivery of such a device is not trivial. Because of the longitudinal sliding freedom of the segments of such devices, alignment of the portions can be compromised if the delivery apparatus is not designed to compensate for positional changes that occur under normal delivery circumstances.

As such, it has been a challenge to develop a delivery assembly that allows for reliable delivery of a segmented medical device in such a way that maintains substantially uniform spacing between the segments of the device.

### BRIEF SUMMARY

The invention may include any of the following embodiments in various combinations and may also include any other aspect described below in the written description or in the attached drawings. This disclosure provides a medical device assembly and methods for delivery of a multi-segment medical device.

In one embodiment, a delivery assembly is provided. The assembly may include a balloon catheter. The balloon catheter may include a catheter body having a first end and extending distally to a second end, the catheter body including a lumen therein. The balloon catheter may also have an expandable balloon disposed circumferentially about the catheter body, the balloon having a proximal portion with a proximal end and a distal portion with a distal end, the proximal end being disposed distal the first end and the distal end being disposed proximal the second end of the catheter body. The assembly may have a retention member disposed at the distal portion of the balloon for allowing an expansion profile of the balloon. The assembly may have a medical implant having a tubular body comprising a plurality of detachable segments, the medical implant being movable between a collapsed configuration and an expanded configuration, the medical implant being disposed about the balloon.

In another embodiment, a delivery assembly is provided. The assembly may include a balloon catheter. The balloon catheter may include a catheter body having a first end and extending distally to a second end, the catheter body including a lumen therein. The balloon catheter may also have an expandable balloon disposed circumferentially about the catheter body, the balloon having a proximal portion with a proximal end and a distal portion with a distal end, the proximal end being disposed distal the first end and the distal end being disposed proximal the second end of the catheter body. The assembly may have a retention member disposed at the distal portion of the balloon for allowing an expansion profile of the balloon. The retention member may be conical and be made of a biocompatible elastomer. The assembly may have a medical implant having a tubular body comprising a plurality of detachable segments, the medical implant being movable between a collapsed configuration and an expanded configuration, the medical implant being disposed about the balloon. The implant may be a segmented stent.

In another embodiment, a method is described. The method may include introducing a delivery assembly to a site to be treated. The assembly may include a balloon catheter. The balloon catheter may include a catheter body having a first end and extending distally to a second end, the catheter body including a lumen therein. The balloon catheter may also have an expandable balloon disposed circumferentially about the catheter body, the balloon having a proximal portion with a proximal end and a distal portion with a distal end, the proximal end being disposed distal the first end and the distal end being disposed proximal the second end of the catheter body. The assembly may have a retention member disposed at the distal portion of the balloon for allowing an expansion profile of the balloon. The assembly may have a medical implant having a tubular body comprising a plurality of detachable segments, the medical implant being movable between a collapsed configuration and an expanded configuration, the medical implant being disposed about the balloon. The method may include expanding the proximal portion free of the retention member by introducing an inflation fluid through the inflation lumen and into the interior of the expandable balloon. The method may include continuing to add the inflation fluid such that the distal portion constrained by the retention member which expands radially after the retention member expands radially first. The method may include contracting the expandable balloon to deploy the medical implant to the target location.

Various additional features and embodiments will become apparent with the following description. The present disclosure may be better understood by referencing the accompanying figures.

### BRIEF DESCRIPTION OF THE DRAWINGS

FIG. 1 depicts a side view of a segmented medical device disposed over an expandable balloon of a balloon catheter in accordance with one embodiment of the present invention;

FIGS. 2A-2C depict side views of an expanded medical device and closeups thereof in accordance with embodiments of the present disclosure;

FIG. 3 is a perspective view of another example of a segmented medical device disposed over an expandable balloon in accordance with another embodiment of the present disclosure;

FIG. 4 is a perspective view of a retention member in accordance with an embodiment of the present disclosure;

FIGS. 5A-5C are side views of a delivery assembly including a medical device in accordance with embodiments of the present disclosure;

FIG. 6 is a perspective view of a portion of a segmented device for use in an assembly according to the principles of the present invention;

FIGS. 7A-7D depict a series of method steps in the inflation of a balloon according to another embodiment of the present invention; and

FIG. 8 depicts a balloon in accordance with one embodiment of the present disclosure.

### DETAILED DESCRIPTION

The present disclosure will now be described more fully with reference to the accompanying figures, which show various embodiments. The accompanying figures are provided for general understanding of various embodiments and method steps. However, this disclosure may be embodied in many different forms. These figures should not be construed as limiting, and they are not necessarily to scale. The following definitions will be used in this application.

“About” or “substantially” mean that two given quantities (e.g. lengths, areas or volumes) are within 10%, preferably within 5%, more preferably within 1%. For example, a first quantity of length can be within 10% of a second length quantity.

“Adjacent” referred to herein is near, near to, or in close proximity with.

“Longitudinally” and derivatives thereof will be understood to mean along the longitudinal axis of the device or device assembly.

The terms “proximal” and “distal” and derivatives thereof will be understood in the frame of reference of a physician using the device. Thus, proximal refers to locations closer to the physician and distal refers to the locations farther away from the physician (e.g., deeper in the patient's vasculature).

The term “biocompatible” refers to a material that is substantially non-toxic in the in vivo environment of its intended use, and that is not substantially rejected by the patient's physiological system (i.e., is non-antigenic).

As used herein, the term “body vessel” means any body passage lumen that conducts fluid, including but not limited to blood vessels, esophageal, intestinal, biliary, urethral and ureteral passages.

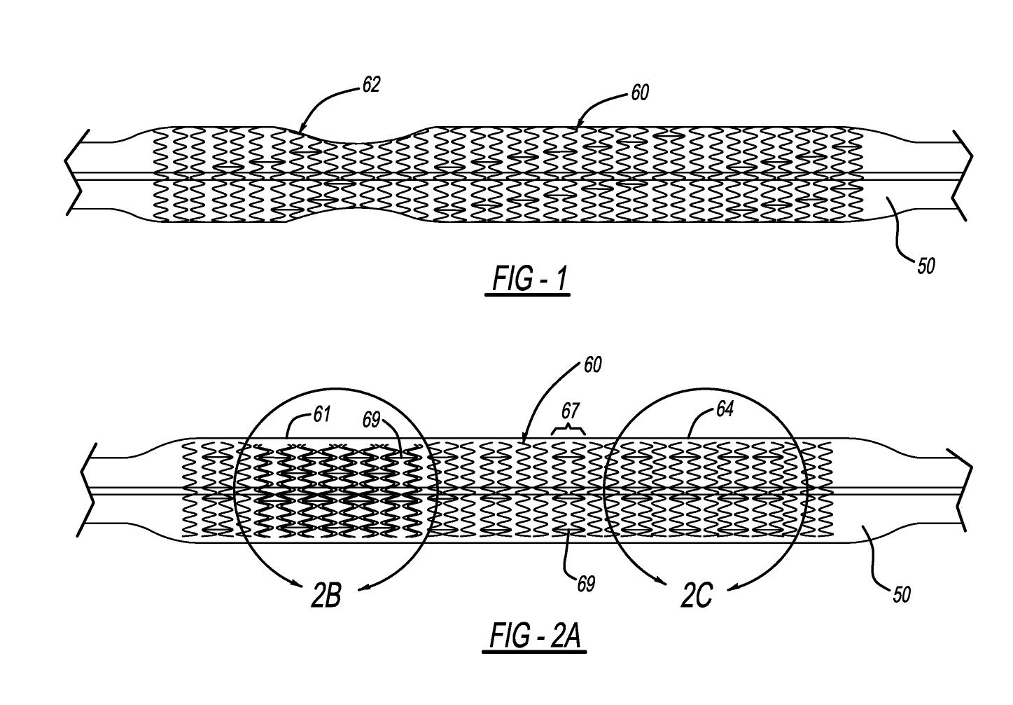

FIG. 1 depicts a medical device 60 according to one embodiment of the present disclosure. For simplicity, the device is depicted schematically. In the case of the device of FIG. 1, the multiportion medical implant 60 is a segmented stent. Segmented stents are multi-portion medical implants. Segmented stents include those described in U.S. patent application Ser. No. 15/335,734, which is incorporated herein by reference in its entirety.

The architecture of a segmented stent may allow for segments of the stent to remain coupled for structural stability during loading and during delivery to a target site, and to uncouple during radial expansion whereby the segments are at least one of longitudinally or circumferentially movable relative to one another after disengagement. Particularly, mating elements forming the interlocking joints described herein that couple adjacent segments in an interlocking relationship and which disengage during expansion of the implant may be provided. For example, stents with these interlocking joints have provided suitable resistance to various mechanical loading from the vessels, such as axial loads (such as compression and tensile, especially resistance to longitudinal stretching), bending loads (such as longitudinal bending), and torque loads. Torque loading and axial loading may occur especially during maneuvering and orienting the stent to the target site. Torque loading and axial loading may also occur during balloon inflation due to uneven expansion of the balloon 50, thereby causing a phenomenon known as “dog-boning,” as shown by region 62 of FIG. 1. Dog-boning results in a radially smaller middle region bounded by two radially expanded regions on either side of the smaller region.

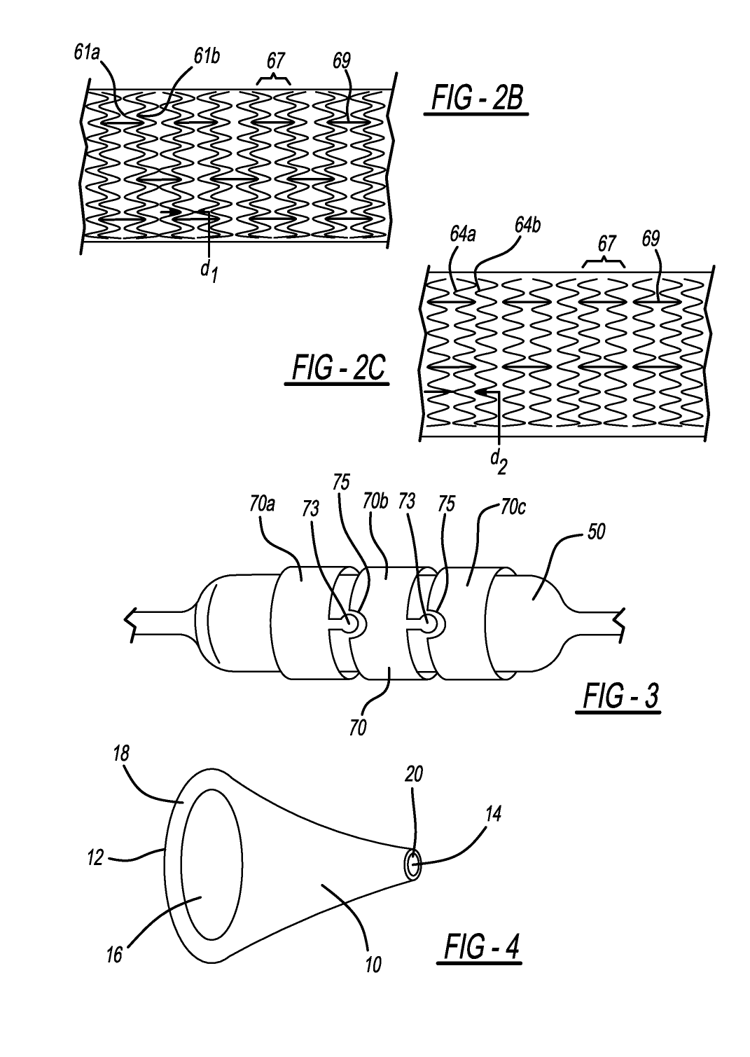

Uneven expansion of the balloon 50, such as under conditions that give rise to dog-boning, can lead to deformation of the fully-expanded device. FIG. 2A provides a view of a segmented stent 60 that has been fully expanded by inflation of a standard balloon 50, yielding uneven spacing of portions of the device 60. The device 60 comprises a plurality of detachable stent segments 67 comprising a plurality of stent rings 62 interconnected by tie-bars 69. The stent segments 67 are coupled together when crimped or disposed over the balloon 50, but are detachable from one another at the time of deployment. The result of uneven inflation on the overall structure of the stent 60 can be seen in FIGS. 2A-2C. For example, the region illustrated in FIG. 2B reveals a misaligned (or crowded) region 61, with distance d₁ between peaks 61a and 61b being closer together than is desirable due to microsliding of the stent segments 67 during inflation of the balloon. The region 61 was at the point of dog-boning during deflation, such that it was at a comparatively reduced diameter. This causes uneven expansion and sliding of various portions of the device 60. Contrarily, region 64 illustrated in FIG. 2C is a properly-spaced portion, in which the distance d₂ between peaks 64a and 64b begin greater than the distance d₁, as inflation of the balloon 50 and therefore expansion of the device 60 was substantially uniform for this region of the implantable device 60.

Turning now to FIG. 3, an interlocking device 70 is portrayed in schematic format. The device 70 has an overall tubular body and, in the illustrated embodiment, includes three interlocking and detachable segments, 70a, 70b, and 70c, which are disposed about balloon 50 such that an inner surface of the tubular body of each segment surrounds the outer surface of the balloon 50. The illustrated embodiment of FIG. 3 has three segments, but other embodiments may have only two segments, or more than three segments. The segments interlock with one another by protrusions 73, which fit into slots 75. The connection between segments 70a, 70b, and 70c may advantageously interconnect in a longitudinal direction, along a line that lies parallel to a longitudinal axis through the device.

In order to modulate or adjust the inflation profile of an expandable balloon of an assembly for delivery and implantation of a segmented device, a retention member 10 as shown in FIG. 4 may be employed. The retention member 10 may be placed over the distal portion of an expandable balloon to form part of a delivery assembly that allows for predictable, consistent, generally unidirectional (proximal-to-distal) inflation of the balloon in such a way that dog-boning and microsliding are minimized. The retention member 10 may have a generally conical or frustoconical shape, having a body that extends from basal end 12 to apical end 14, the body defining a lumen or cavity 16 through the retention member 10, terminating in opening 20. The basal end 12 may include a lip 18 that surrounds the opening to lumen 16.

The retention member 10 is preferably made of a resilient material, one that will resist expansion as the balloon inflates to a certain point, and then finally expands as the remainder of the balloon has filled with an inflation fluid. The retention member may be made of a biocompatible material, such as a biocompatible elastomer, including for example a rubber or a polymer. The retention member 10 may have a radial force resistance to being radially expanded equal to, or greater than, the medical implant crimped over the balloon, such that it expands last when the balloon is inflated.

In one embodiment, the retention member 10 may be a separate component formed independently from the remainder of the delivery assembly. The retention member 10 may then be attached to the assembly such as with an adhesive. In another embodiment, the retention member may be placed over the distal portion of the balloon and may remain in place due to its elasticity, or its tendency to produce an inward radial force. In certain embodiments, the balloon 50 may have a textured surface such that the retention member does not slide off of the balloon. Such a textured surface may include surface roughening or microbristles.

In another embodiment, the retention member may be unitary with the balloon. One example of a unitary retention member is, for example, a coating applied to the outer surface of the distal portion of a balloon. The coating could be applied by any conventional method, such as spray coating or dip coating, and may be made of the same material as the balloon or a different material. In another embodiment, the unitary retention member may be a thickened balloon wall, as will be described in accordance with the description of FIG. 8.

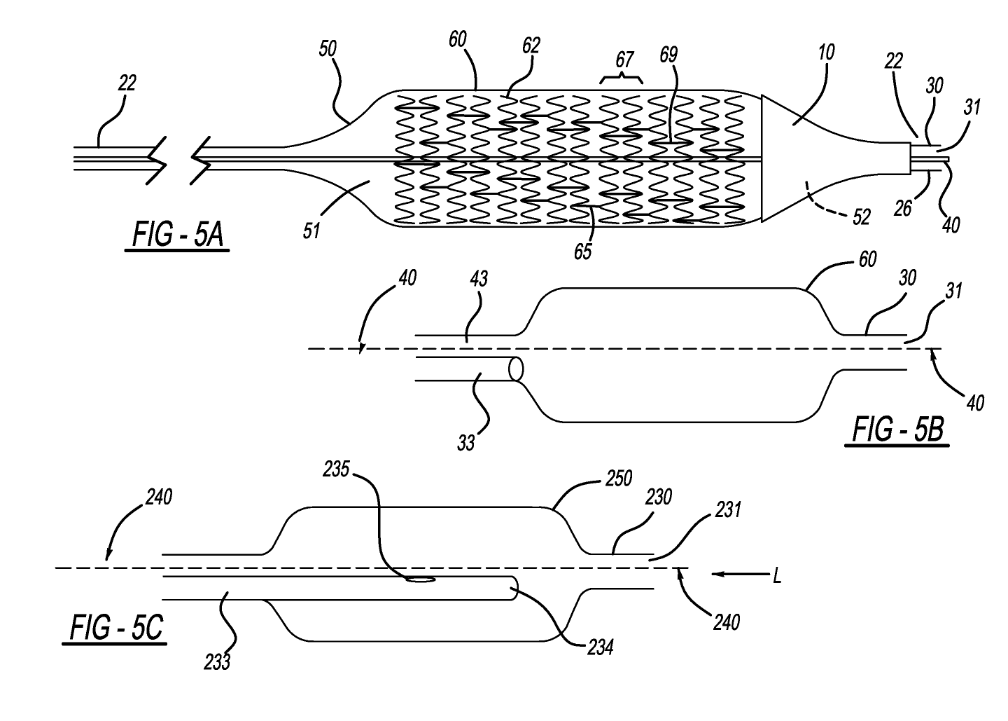

FIG. 5A depicts an assembly including a balloon catheter 22, which includes a catheter shaft 23 and a balloon 50. The balloon 50 is expandable, and can be inflated by the addition of an inflation fluid, and deflated by the withdrawal of inflation fluid. The catheter has a shaft 23 extending along a longitudinal axis L from a first end 24 to a second end 26. The catheter shaft 23 can include a lumen 31, which may be at least one of an inflation lumen 33 (see FIG. 5B) to deliver an inflation fluid and/or a wire guide lumen 43 for receiving a wire guide 40. The balloon 50 may be mounted on the catheter shaft 23 and may be in fluid communication with the inflation lumen 33.

As depicted in FIG. 5A, the assembly may include a balloon 50 that has a free end (that is, not constrained by a retention member 10) at its proximal portion 51 and a restrained end at its distal portion 52, with retention member 10 surrounding said distal portion 52. The retention member 10 may also extend over a portion of the body 23 of catheter 22 to further secure the retention member 10 to the rest of the assembly. In one embodiment, the retention member may be adhered to the balloon, to the catheter body 23, or to both. The basal end of the retention member 10 may overlie the distal portion 52 of balloon 50, and the apical end of the retention member 10 may overlie the body of the catheter 22.

A number of luminal configurations for the balloon catheter 22 are possible. One lumen design is shown in FIG. 5B. In this embodiment, lumen 31 is subdivided into wire guide lumen 43, which contains wire guide 40 and allows it to be substantially coincident with longitudinal axis L of the assembly, and inflation lumen 33, which terminates in an open end just proximal, or within, the interior of balloon 50. Inflation lumen 33 is in fluid communication with the interior of balloon 50 and extends to the proximal end 24 of catheter 22, where it may be connected to a reservoir of inflation fluid for inflating the expandable balloon 50.

In another embodiment, the lumen configuration of a catheter assembly in accordance with another embodiment of the present invention is illustrated. In this embodiment, inflation lumen 233 extends further into the interior of balloon 250, with a side port 235 providing fluid communication between the lumen of the inflation catheter and the interior of the balloon. The inflation lumen also includes a closed end 234 such that fluid can only be injected through the side port 235. In a related embodiment, the closed end 234 may be located distal the distal end of balloon 250.

The balloon 50 of a catheter assembly may be fluid-tight so as to avoid leaking of inflation fluid. Typically, the inner surfaces of the balloon 50 is sealably attached to the catheter shaft 23 to prevent leakage of fluid. Means of sealing the balloons may include, for example, heat sealing, using an adhesive to form the seal, forced convection heating, radio frequency heating, ultrasonic welding, and laser bonding. Shrink tubing can be used as a manufacturing aid to compress and fuse the balloon to the catheter shaft defining either of the wire guide lumen 43 and the inflation lumen 33. If the catheter shaft 23 has an outer coating, the balloon 50 can be bonded to the coating or directly to the catheter shaft.

Optionally, the delivery assembly can include radiopaque material to provide a means for locating the multiple-balloon catheter within a body vessel. For example, the catheter shaft 23 can include one or more marker bands annularly disposed around the outside of the catheter shaft within the balloon 50. If desired, radiopaque bands can be added to the catheter shaft. Radiopaque marker bands can be used by a clinician to fluoroscopically view and locate the distal portion of the multiple-balloon catheter at a point of treatment within a body vessel. The radiopaque material can comprise any suitable opacifying agent.

In some embodiments, the delivery assembly may not have an outer sheath covering the balloon 50 and the implant 60 crimped over the balloon. Such an arrangement may be advantageous as it reduces the profile of the delivery assembly. In other embodiments, an outer sheath may surround the delivery assembly, which may be as depicted in FIG. 5A.

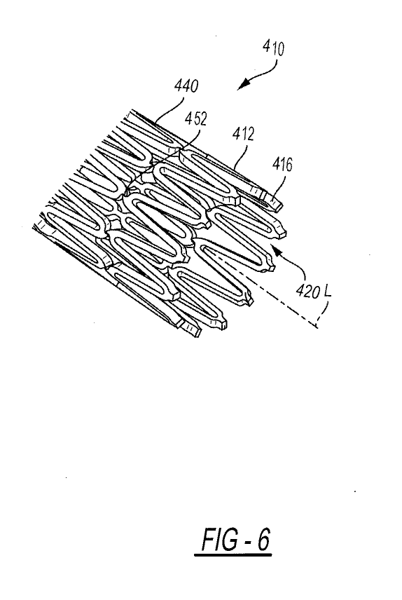

In some embodiments, the medical implant to be delivered may be a segmented stent. FIG. 6 depicts a portion of a stent 410 which may be used in conjunction with or as part of a delivery assembly as disclosed herein. The stent 410 may include a number of struts 416 arranged into stent rings 412. The interlocking joint design of the stent 410 may maintain axial and circumferential engagement to inhibit the stent segments from losing their relative orientation to one another during delivery and may minimize partial expansion events like dog-boning. Mating elements 452 forming the interlocking joints 430 may also be configured to disengage in at least one of the axial and circumferential directions to avoid mating elements jamming during release. When the stent is implanted in a body vessel, the stent architecture of the now discrete axial stent segments separated from one another at deployment may provide at least one of the following: more uniform radial expansion; suitably high radial force and high circumferential compression resistance to hold lesion out of vessel lumen; suitable longitudinal flexibility and conformability for tortuous vessels; and greater bending and longitudinal compression from vessel contributing high-level motion environments. In addition, the mating elements 452 forming the interlocking may be micro-mating elements or may be as small as possible (such as less than the width of a strut 418) to minimize body tissue interaction, yet perform at least one of the functions as detailed above.

The stent 410 includes a plurality of stent segments coupled together by a plurality of interlocking joints 430. In one example, the axial stent segments comprise one or more ring structures 412 disposed axially relative to one another along a longitudinal axis L. The stent 410 is defined as a tubular body defining a lumen disposed about the longitudinal axis L between a first stent end (not shown) and a second stent end 420. The tubular body includes an exterior surface to contact the body vessel wall and an opposite, interior surface facing the lumen. Adjacent ring structures 412 (for example, a first ring structure 412A and a second ring structure 412B) may be coupled to one another by the interlocking joints 430. Also shown is that adjacent ring structures 412 may be interconnected by a plurality of tie bars.

The ring structure 412 has a substantially circular ring shape comprising an undulating arrangement of interconnected unit stent struts 418. The undulating arrangement may be also defined also as a serpentine or zigzag pattern. The stent struts 418 may be connected to one another at opposite joints to define a first series of apices disposed axially opposite and circumferentially offset to a second series of apices. The stent segments of the stent 410 may be comprised of ring structures 412, axial stent members, or combination of the two. Depending on the location of the axial stent member or ring structure 412, the tie bars and/or interlocking joints 430 may be omitted from the corresponding inner apices and/or outer apices. For example, when the axial stent member or ring structure 412 is disposed along the outermost extreme axial ends of the stent 410, the outer apices may not include any tie bar or interlocking joint. Further, depending on the environment and length of the point of treatment, a single ring structure 412 may be used along the end of the stent 410, rather than the axial stent member comprising of two or more ring structures 412.

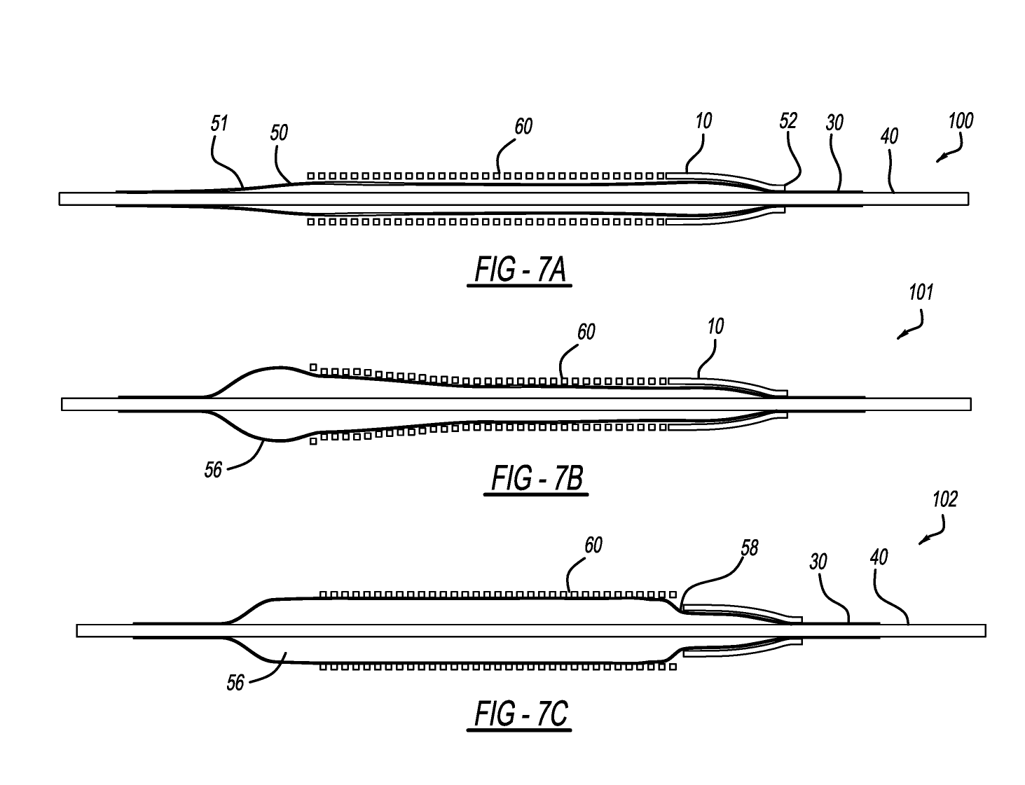

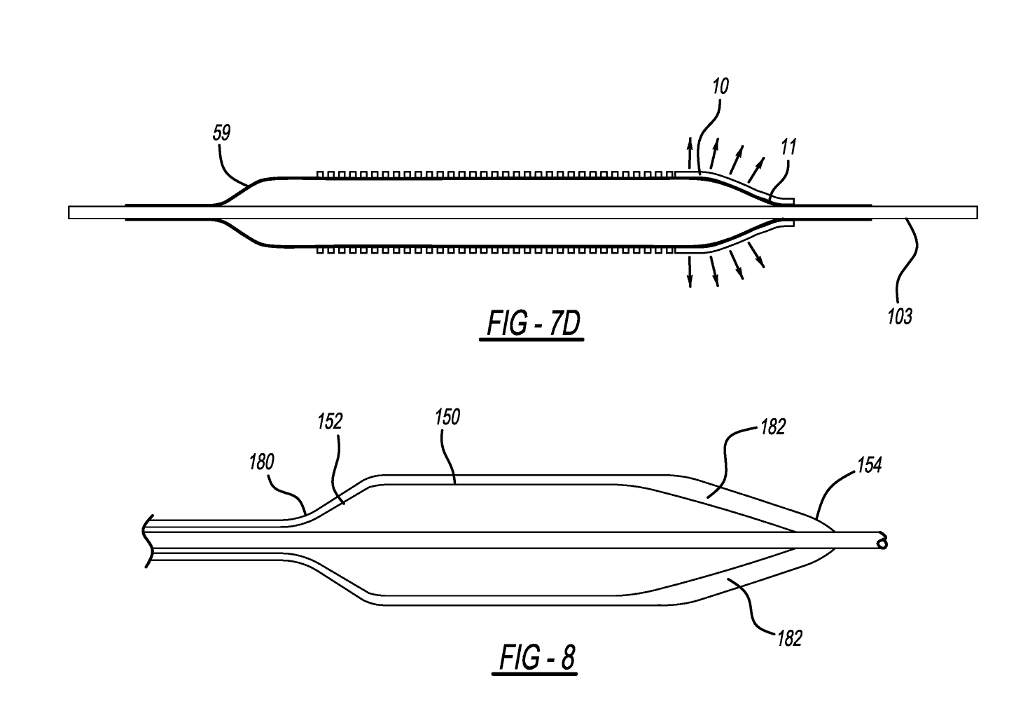

FIG. 7 illustrates a method of using the delivery assembly in conjunction with an implantable medical device as described. It will be appreciated that the steps illustrated are exemplary of a single embodiment of the invention and may be practiced as presented, or in conjunction with further method steps.

First step 100 is illustrated in FIG. 7A. In this step, the assembly has been advanced to the treatment site within the body vessel of the patient to be treated. At this time, the assembly includes a balloon catheter 22 with a deflated balloon 50 disposed about catheter body 30 and extending between proximal portion 51 and distal portion 52. The distal portion 52 is constrained within restraining member 10, which overlies the exterior of balloon 50. The radially expandable medical device 60 is disposed about balloon 50. Wire guide 40 runs through a lumen (not illustrated).

In a second step 101, inflation fluid has begun to flow into the interior of the balloon 50 to create a partially-inflated balloon having partially expanded or inflated proximal portion 56. Inflation at the proximal portion 51 is possible because proximal portion 51 is free from a constraining member, whereas distal portion 52 is held radially contracted by retention member 10. Similarly, the proximal-most portion of radially expandable device 60 has begun to radially expand because of the radial expansion of proximal portion 51 of the balloon.

In a third step 102, illustrated in FIG. 7C, more inflation fluid has been introduced into the interior of balloon. Fully-inflated (or fully-expanded) proximal portion 57 is shown in this figure, proximal of a still-constrained distal portion 58. Enough fluid has been introduced to produce partially-inflated distal portion 58. However, the retention member 10 remains in a constrained configuration, thereby causing the distal-most portion of the balloon 50 to remain in a relatively contracted configuration. Taken together, FIGS. 7B and 7C demonstrate how inflation has largely proceeded in a proximal-to-distal direction, or in a unidirectional fashion (that is, in a single direction portion-wise, proximal to distal).

FIG. 7D shows a step 103 in which sufficient inflation fluid has been introduced into the balloon 50 to cause the balloon to fully inflate 59. The influx of fluid has, at this point, overcome the radially-inward constraining force of retention member 10, thus causing the retention member 10 to adopt expanded configuration 11. The device 60 at this point is fully expanded, and due to the unidirectional expansion profile, has been expanded substantially evenly, minimizing microsliding and dog-boning. To deploy the device, the balloon 50 may then be deflated by withdrawing inflation fluid proximally through an inflation lumen of the catheter, and the assembly can be withdrawn from the body of the patient. As the delivery assembly has its balloon deflated, the interconnected segments of device 60 may disengage longitudinally from one another, and be implanted within the body vessel.

It will be appreciated that any balloon, assembly, and catheter design as disclosed herein may be employed to affect the method of FIG. 7.

FIG. 8 is an illustration of another embodiment of a balloon 150 which has a unitary retention member formed thereon. The balloon 150 has a distal portion 154 which resists inflation as It has a thicker wall 182 than the thickness of the wall 180 at proximal portion 152. This causes substantially similar expansion profile as in the balloon in which the retention member is a separately-formed element, and the distal portion will inflate after the proximal portion.

It should be understood that the foregoing relates to exemplary embodiments of the disclosure and that modifications may be made without departing from the spirit and scope of the disclosure as set forth in the following claims. While the disclosure has been described with respect to certain embodiments it will be appreciated that modifications and changes may be made by those skilled in the art without departing from the spirit of the disclosure.

1. A delivery assembly comprising:
a balloon catheter comprising:
a catheter body having a first end and extending distally to a second end, the catheter body comprising a lumen therein, and
an expandable balloon disposed circumferentially about the catheter body, the balloon having a proximal portion with a proximal end and a distal portion with a distal end, the proximal end being disposed distal the first end and the distal end being disposed proximal the second end of the catheter body;
a retention member disposed at the distal portion of the balloon for allowing an expansion profile of the balloon; and
a medical implant having a tubular body comprising a plurality of detachable segments, the medical implant being movable between a collapsed configuration and an expanded configuration, the medical implant being disposed about the balloon.

2. The assembly of claim 1, wherein the retention member comprises a conical cap having an apical end and extending to a basal end, the basal end surrounding the distal portion of the balloon.

3. The assembly of claim 2, wherein the conical cap comprises an elastic material.

4. The assembly of claim 2, wherein the conical cap surrounds a portion of the catheter body distal the expandable balloon.

5. The assembly of claim 4, wherein the conical cap is attached to the catheter body by an adhesive.

6. The assembly of claim 4, wherein the conical cap is attached to the catheter body by a radial tension.

7. The assembly of claim 2, wherein the conical cap is attached to the expandable balloon by an adhesive.

8. The assembly of claim 2, wherein the expandable balloon comprises an exterior surface having a plurality of microbristles for retaining at least one of the medical implant and the conical cap.

9. The assembly of claim 1, wherein the retention member comprises a coating formed over the distal portion.

10. The assembly of claim 1, wherein the distal portion of the expandable balloon comprises a thickened balloon wall.

11. The assembly of claim 1, wherein the retention member comprises a biocompatible elastomer.

12. The assembly of claim 1, wherein the retention member has a radial force resistance at least substantially equal to that of the medical implant.

13. The assembly of claim 1, wherein the medical implant comprises an interlocking stent.

14. An assembly for delivering a medical implant, the system comprising:
a balloon catheter comprising:
a catheter body having a first end and extending distally to a second end, the catheter body comprising a lumen therein, and
an expandable balloon disposed circumferentially about the catheter body, the balloon having a proximal portion with a proximal end and a distal portion with a distal end, the proximal end being disposed distal the first end and the distal end being disposed proximal the second end of the catheter body;
a retention member comprising a conical cap disposed about the distal portion of the balloon, the conical cap comprising a biocompatible elastomer, the retention member allowing for an expansion profile of the balloon; and
a medical implant comprising a stent having a tubular body comprising a plurality of detachable segments, the medical implant being movable between a collapsed configuration and an expanded configuration, the medical implant being disposed about the balloon.

15. A method of using a delivery assembly, the method comprising:
introducing the delivery assembly to a target location of a body vessel, the delivery assembly comprising:
a balloon catheter comprising:
a catheter body having a first end and extending distally to a second end, the catheter body comprising an inflation lumen therein, and
an expandable balloon disposed circumferentially about the catheter body, the balloon having a proximal portion with a proximal end and a distal portion with a distal end, the proximal end being disposed distal the first end and the distal end being disposed proximal the second end of the catheter body;
a retention member disposed at the distal portion of the balloon for allowing an expansion profile of the balloon; and
a medical implant, mounted over the expandable balloon;
expanding the proximal portion free of the retention member by introducing an inflation fluid into the interior of the expandable balloon such that the retention member prevents the distal portion from expanding prior to full expansion of the proximal portion; and
expanding the distal portion and the retention member after full expansion of the proximal portion.

16. The method of claim 15, wherein the medical implant is movable between a radially collapsed configuration and a radially expanded configuration, the medical implant having a tubular shape and comprising a plurality of detachable segments, the medical implant being mountable over the expandable balloon, such that the plurality of detachable segments are interconnected when the medical implant is mounted over the expandable balloon.

17. The method of claim 15, wherein the medical implant comprises a segmented stent.

18. The method of claim 15, wherein the retention member comprises a conical cap having an apical end and extending to a basal end, the basal end surrounding the distal portion of the expandable balloon.

19. The method of claim 15, wherein the retention member comprises a biocompatible elastomer.

20. The method of claim 15, wherein the conical cap is attached to the catheter body by an adhesive.
